(19) United States
(12) Patent Application Publication
(10) Pub. No.: US 2014/0220667 A1
(43) Pub. Date: Aug. 7, 2014

(54) Device for pathogen detection

(71) Applicants: Minhaz Uddin Ahmed, (US); Mohammed Zourob, (US); Mohammadali Safavieh, (US)

(72) Inventors: Mohammadali Safavieh, Montreal (CA); Minhaz Uddin Ahmed, St-Leonard (CA); Mohammed Zourob, Bedfordshire (GB)

(73) Assignees: Mohammadali Safavieh, Montreal (CA); Mohammed Zourob, Bedfordshire (GB); Minhaz Uddin Ahmed, Montreal (CA)

(21) Appl. No.: 13/987,392

(22) Filed: Jul. 22, 2013

Related U.S. Application Data
(60) Provisional application No. 61/678,792, filed on Aug. 2, 2012.

(51) Int. Cl. C12Q 1/68

(52) U.S. Cl. CPC C12Q 1/689
USPC 435/287.2

(57) ABSTRACT

There is provided a portable device for detecting pathogens in a sample, the detection being based on an assay involving one or more reagents and heat. The device comprises a flexible substrate having a plurality of spaced apart reservoirs along its length, each reservoir adapted for receiving reagents and the sample, the substrate being secured to a pair of spaced apart reels and movable there between by rolling. Also, the device comprises heating means which can be secured to one of the two reels.

### FIELD OF THE INVENTION

This application claims the benefit of U.S. provisional Application No. U.S. 61 678 792, filled Aug. 2, 2012, the content of which incorporated in their entirely herein. The invention relates generally to pathogens detection. More specifically, the invention relates to a device for detecting pathogens in a sample based on assays involving reagents and heat. The invention also relates to a device for use in the diagnosis of diseases including medical conditions caused by food poisoning, biological contamination and bioterrorism.

### BACKGROUND OF THE INVENTION

Pathogens such as bacteria, viruses, fungi and other microorganisms have a profound impact on humans and animals and are the cause of various infectious diseases. Infectious diseases account for nearly 40% of the total 50 million deaths that occurred annually, both in developed and developing countries. Detection of these pathogens plays an important role in the global health care system, as it allows for appropriate timely intervention. Devices for detecting pathogens are known in the art. However, most of those devices usually embody techniques that make the devices not suitable for use in places with limited resources. Also, the devices may not be reliable due to low sensitivity and high levels of false negative bands. Such devices include for example devices for bacteria detection based on enzymatic linked immunoassay (ELISA). The drawbacks of those techniques have been overcome by nucleic acid (NA) amplification techniques.

Devices for detecting pathogens can be used in the diagnosis of diseases. Techniques based on molecular biology such as NA amplification are well-established rapid methods that do not require cultivation or pre-enrichment of pathogens if the DNA sequence information already exists in known databases [1]. Despite these advantages, it is usually still necessary to perform culturing techniques, which take a long time, between 6 hours to several days besides, and need skilled personnel and high precision laboratories [2]. This delay has a huge impact on mortality and negative effects on patients. There is therefore a need for rapid, sensitive, portable and easy to use devices for detecting pathogens.

Lab on chip (LOC) devices allow for point of care (POC) diagnoses, yet only a few of such devices can process raw samples and even a fewer of these devices are suited for limited-resources settings. Besides, most of the fabrication of microfluidic devices needs to be performed in highly equipped clean rooms, and use expensive reagents and polymers [1]. Also, nucleic acid amplification techniques need sample preparation and preprocessing for extraction and purification of the DNA. Therefore, building a LOC device based on NA amplification results in an increase of the cost, due to the complexity of the design required [3].

Aside from cost and complexity of the device, most of the current LOC devices allow for the analysis of only a limited number of samples. This constitutes a limit for their efficient use in highly populated areas such as health care centers and hospitals. Accordingly, there is a need for a pathogens detection device based on nucleic acid amplification, which is simple to use and which allows for the process of many samples at the same time. It is also desirable that such device provide for a high precision.

One of the most versatile bacteria in the world is Escherichia Coli [4]. It can exist either as beneficial bacteria living in the digestive tract [5] or as a poisonous pathogen in the food and environment. E. coil can cause many diseases in humans such as meningitis and Urinary Tract Infection (UTI) [6]. There is a need for a device that allows for an efficient detection of E. coli or any other pathogens. Preferably, the device can be manufactured in a laboratory setting. If the targeted pathogen is E. coli, LAMP technique for E. coli detection [7] may be used. The device may also be used with any other amplification techniques suitable for pathogens detection such as polymerase chain side reaction (PCR).

### SUMMARY OF THE INVENTION

The inventors have designed and manufactured a simple, easy to use and efficient LOC device for the detection of pathogens. The device allows for POC diagnoses. The device can be manufactured in a laboratory setting involving manufacturing techniques such as hot embossing or roll-to-roll printing. The device comprises a flexible plastic roll in a cassette format. The device may use LAMP technique or any other suitable amplification technique for pathogens detection. The device allows for the analysis of hundreds of sample at the same time (high throughput detection of pathogens). Moreover, the device is portable. The process for the manufacture of the device according to the invention is set forth herein below. Use of the device is also outlined.

Accordingly, the invention thus provides according for the following:

(1) A portable device for detecting pathogens in a sample, wherein the detection is based on an assay involving one or more reagents and heat.

(2) A portable device for detecting pathogens in a sample, comprising a flexible substrate having a plurality of spaced apart reservoirs along its length, each reservoir adapted for receiving reagents and the sample, the substrate being secured to a pair of spaced apart reels and movable there between by rolling, the device further comprising heating means, preferably secured to one of the two reels, for transmitting heat to the reservoirs, wherein the detection is based on an assay involving the reagents and heat.

(3) A device for detecting pathogens in a sample, comprising a flexible substrate having a plurality of spaced apart reservoirs along its length, each reservoir having one or more reagents therein and adapted for receiving the sample, the substrate being secured to a pair of spaced apart reels and movable there between by rolling, the device further comprising heating means, preferably secured to one of the two reels, for transmitting heat to the reservoirs, wherein the detection is based on an assay involving the reagents and heat.

(4) A device according to any one of (1) to (3) above, wherein the assay is selected from loop-mediated isothermal amplification (LAMP) assay, colorimetric assay, electrochemical assay, fluorescence-based assay and assay involving viscosity measurement.

(5) A device according to any one of (1) to (4) above, wherein the assay allows for detection of amplification in real time.

(6) A device according to any one of (1) to (5), wherein the pathogen is gram-negative or gram-positive bacteria, a virus, fungi or any other genomic DNA, RNA or ssDNA.

(7) A device according to any one of (1) to (6), wherein the pathogen is Escherichia coli or Staphylococcus aureus.

(8) A device according to any one of (1) to (7), wherein the reagent comprises LAMP reagent, hydroxynaphtol blue (HNB), calcein, a redox molecule such as osmium or ruthenium complexes.

(9) A device according to any one of (1) to (8), which allows for high throughput detection of pathogens.

(10) A device according to any one of (1) to (9), wherein the sample is from a human, an animal, food, a food environment, an office environment or any other environment.

(11) A device according to any one of (1) to (10), further comprising heat control means for controlling heat transmitted to the reservoirs.

(12) A device according to any one of (1) to (11), wherein heat transmitted to the reservoir is collected from other sources such as sun, solar-cells or any other heat-generating materials or systems.

(13) A device according to any one of (2) to (12), wherein the reservoirs are closed reservoirs.

(14) A device according to any one of (2) to (13), wherein the flexible substrate including the closed reservoirs constitutes a unitary body, or the flexible substrate further comprises a separate piece for covering the reservoirs.

(15) A device according to (14), further comprising means for providing the separate piece for covering the reservoirs.

(16) A device according to 15, wherein the means for providing the separate piece for covering the reservoirs comprises a reel system which has one or more reels.

(17) A device according to any one of (2) to (16), wherein the flexible substrate is made of a material which is a plastic material made of polyethylene or any other plastic materials.

(18) A device according to any one of (14) to (17), wherein the separate piece for covering the reservoirs is made of a plastic material made of polyethylene, poly acetate, polyvinylchloride (PVC) or polyethylene terephthalate (PET).

(19) A device according to (15) or (16) above, wherein the separate piece is a cello-tape.

(20) A device according to any one of (2) to (19), which is run using sun, solar cells or any other heat-generating materials or systems.

(21) A method for diagnosing a disease in a human or an animal, comprising using the device according to any one of (1) to (20), wherein the sample is from the human or animal.

(22) A method according to (21), wherein sample processing such as cell lysis or genetic materials fishing is performed prior to the use of the device.

(23) A method according to (21) or (22), wherein the disease is a disease caused by the pathogens.

(24) A method according to any one of (21) to (23), wherein the disease is meningitis, urinary tract infection (UTI), a medical condition caused by food poisoning, biological contamination or bioterrorism.

### BRIEF DESCRIPTION OF THE FIGURES

FIG. 1 illustrates the device according to the invention for colorimetric detection.

FIG. 2 illustrates the device according to the invention for real time electrochemical detection.

FIG. 3 illustrates the process of manufacturing the flexible substrate of the device according to the invention of colorimetric assay.

FIG. 4 illustrates the process of manufacturing the flexible substrate of the device according to the invention of electrochemical assay.

FIG. 5 illustrates results of an assay involving use of the device according to the invention for E. coli bacteria using HNB dye.

FIG. 6 illustrates results of another assay for S. aureus bacteria detection involving uses of the device according to the invention using Calcein fluorescent detection.

FIG. 7 illustrates the process of electrochemical real-time assay.

FIG. 8 illustrates an embodiment of the device according to the invention for real-time electrochemical detection of E. coli bacteria.

FIG. 9 illustrates an embodiment of the device according to the invention for real-time electrochemical detection of S. aureus bacteria.

### DETAILED DESCRIPTION OF PREFERRED EMBODIMENTS OF THE INVENTION

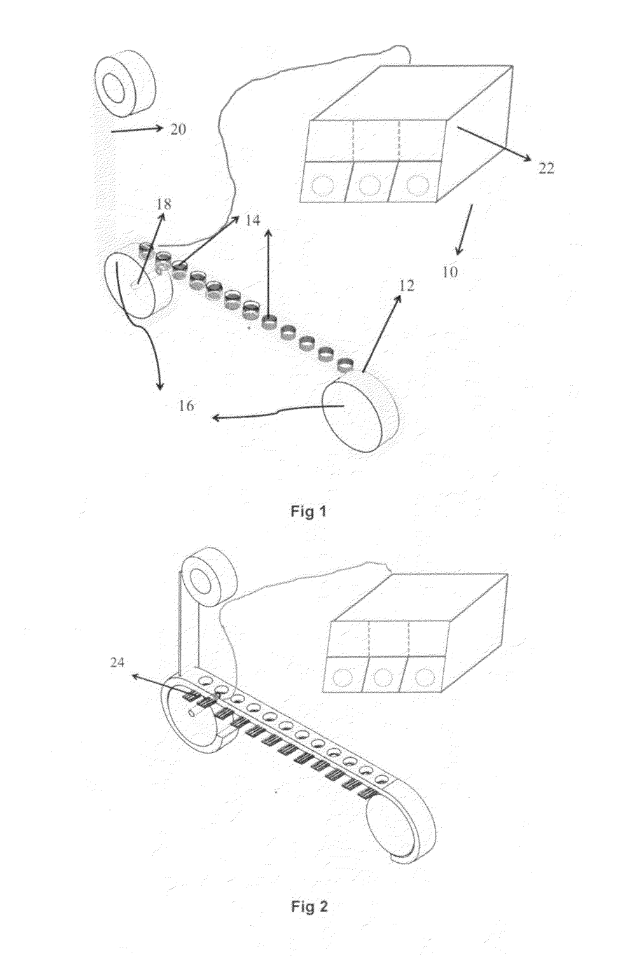

Referring to the figures, the inventors have designed and manufactured a portable device 10 for detecting pathogens in a sample. The detection is based on an assay involving one or more reagents and heat. The device 10 comprises a flexible substrate 12 having a plurality of spaced apart reservoirs 14 along its length as illustrated in FIG. 1. Each reservoir is adapted to receiving reagents and the sample to be analyzed. The reservoirs 14 can be closed reservoirs provided in the flexible substrate 12 which is manufactured as illustrated in FIG. 1. The flexible substrate 12 is made of a material that allows for the reagents and sample to be deposited therein while they remain closed. Such material includes for example a plastic material made of polyethylene or any other plastic material. The flexible substrate 12 including the closed reservoirs can be a unitary body. Alternatively, a cover 20 for the reservoirs can be provided as a separate piece. Also, the flexible substrate 12 can be provided with reservoirs preloaded with the reagents and ready for the sample to be deposited therein.

In embodiments of the invention, the flexible substrate 12 is secured to a pair of spaced apart reels 16 as illustrated in FIG. 1. The flexible substrate is movable between the two reels 16 for example by rolling. The device further comprises heating means for transmitting heat to the reservoirs 14. Heat transmitted to the reservoirs is obtained from electrical heater 18 and heat controller 22. The heating means can be secured to one of the two reels 16. In the device according to the invention, detection is based on an assay involving reagents and heat.

In embodiments of the invention, the cover 20 for the reservoirs 14 may be provided using other suitable means. As will be understood by a skilled person, such reel system may comprise one or more reels. The cover 20 can be a cello-tape.

In embodiments of the invention, the assay may be any nucleic acid amplification techniques such as PCR or loop-mediated isothermal amplification (LAMP) assay, colorimetric assay, electrochemical assay, fluorescence-based assay and assay involving viscosity measurement. Such assay can use wireless sensors. Also, such assay can allow for detection of amplification in real time. For example, FIG. 3 illustrates the results obtained in the detection of E. coli in a sample using the device according to the invention. The pathogen may be any other gram negative or positive bacteria, a virus or fungi. Also, the assay can allow for the detection of any genomic DNA, RNA or ssDNA.

In embodiments, the reagent comprises LAMP reagent, hydroxynaphtol blue (HNB), calcein or any suitable redox molecules such as ruthenium and osmium complexes. For example, in the experiment, the results of which are illustrated in FIG. 3, LAMP reagent, HNB and calcein were used.

The device according to the invention allows for high throughput detection of pathogens. Various types of sample can be analyzed using the device. For example the sample can be from a human, an animal, food, a food environment, an office environment or any other environment.

In embodiments, the device according to the invention further comprises heat control means for controlling heat transmitted to the reservoirs. This feature allows for the control of the temperature in the reservoirs. It will be understood that depending on the targeted pathogen and the assay technique used, the required temperature in the reservoir may be different.

The device according to the invention can be used for diagnosing a disease in a human or an animal. Accordingly, the sample will be a sample from the human or animal. Such disease may be for example meningitis, urinary tract infection (UTI) or a medical condition caused by food poisoning, biological contamination or bioterrorism.

Experimental Set Up

Cassette is composed of two aluminum reels as collector and provider of plastic roll. The provider reel acts as a heater to provide 66° C. temperature for LAMP amplification. 25 μL of samples are loaded into each reservoir. Further, the plastic roll is swathed into the collector reel to heat. After certain amount of amplification time, the roll is extended and the result of bacterial detection is visualized by naked eye. FIG. 1 shows the schematic of the cassette.

For S. aureus bacteria detection, 5 μL of samples are loaded into each reservoir and covered with tape. Further, the plastic roll is swathed into the collector reel to heat at 90 C for 5 min. Then it rolled back and open the cover again and add 20 μL of LAMP solution and covered with tape. The plastic roll is rolled into the collector reel and after 1 hr amplification time the roll is extended and result of bacteria detection is observed by naked eye.

LAMP Assay

LAMP protocol is used to detect Tuf gene in E. coli bacteria as well as Mcat gene of Staphylococcus aureaus. The protocol has been optimized in order to be compatible with our primers. The primers sequences has been mentioned previously in [9]. 20 μL of master mix composed of 2.5 μL of 10× Thermopol Buffer, 3.2 μL of 0.6 μM Betaine, 1.25 of 5 mM MgSO₄, 1 μL of Bst Polymerase (1600 units), 8000 U/ml, 0.6 mM concentration 0.6 μl of 0.6 μM dNTP, 0.15 μl of 120 μM HNB, 0.25 μl of 0.2 μM outer primers (F3, B3), 2.0 μl of 1.6 μM inner primers (FIP, BIP) and 1.0 μl of 1.0 μM loop primers (Loop F, Loop B). The primers sequences has been used previously [9] for detection of Tuf gene of E. coli and Mcat gene of S. aureus, respectively.

FIG. 1 illustrates the cassette or device 10 as POC device. The flexible substrate 12 is attached to the reels 14. Samples are loaded to the reservoirs and are covered by a tape. Two reels 16 provide heat for amplification and collecting of the substrate. Illustrates the cassette 10, and a heat controller 22, electric heater 20 and collector reel illustrates the flexible substrate 12. By turning the flexible ribbon into the collector reel, the tape 20 covers all the samples in order to protect them from evaporation.

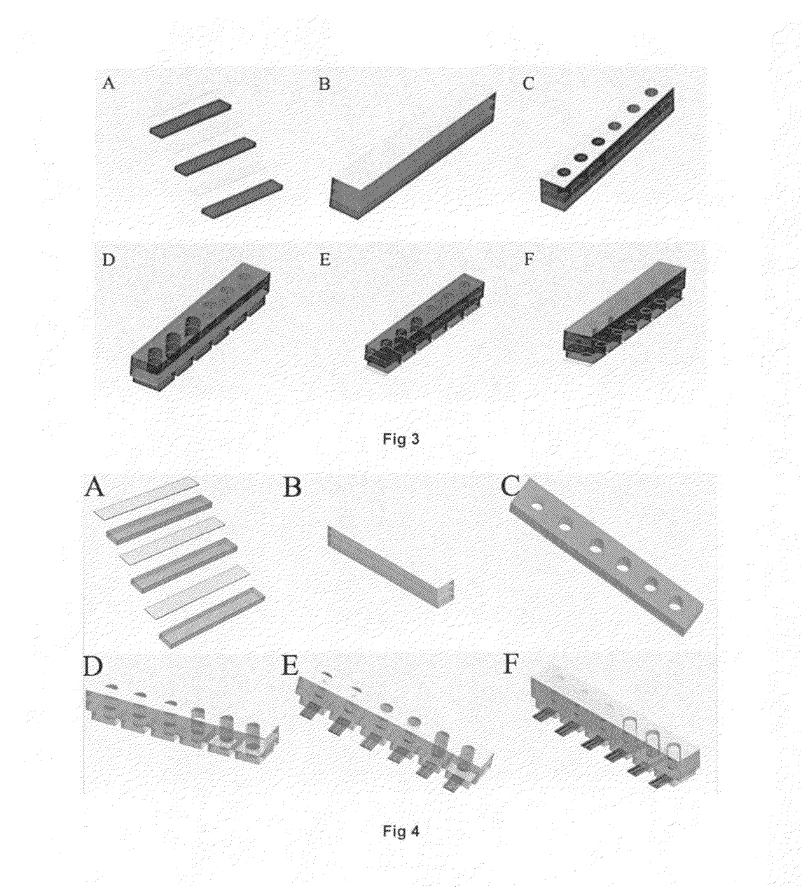

FIG. 2 illustrates the cassette. The flexible ribbon 20 with flexible screen-printed electrodes 24 are attached at the bottom of each reservoirs and were attached to the reels. Samples are loaded and covered by tape. Each electrode is connected to the potentiostat to detect the amplicon in a real time analysis.

Fabrication Process

In the fabrication process, we provide a series of 35 μL-reservoirs for implementation of nucleic acid amplification. Polyethylene strips have been used as a substrate. Three layers of polyethylene strips were cut and assembled by hand and attached on top of each other using double-sided Kapton® tape. 35 μL-reservoirs have been structured on the substrate. One-sided tape is attached to complete the reservoirs. Once the samples have been added to the reservoirs, they will be covered by tape to protect the samples and to prevent evaporation.

FIG. 3 shows the fabrication process of the flexible substrate in detail. (A) Flexible substrate consists of three layers of polyethylene ribbons, which was attached using double-sided tape. (B) The layers were attached on top of each other. (C) Punching the substrate to form reservoirs. (D) Removing the redundant to enhance bending and flexibility. (E) Attaching the plain paper at the bottom of each reservoir. (F) Apply the samples and cover the reservoirs using the tape.

FIG. 4 shows the schematic of the fabrication process. (A) Flexible substrate consists of three layers of polyethylene ribbons, which was attached using double-sided tape. (B) The layers were attached on top of each other. (C) Punching the substrate to form reservoirs. (D) Removing the redundant to enhance bending and flexibility. (E) Attaching the flexible carbon electrode at the bottom of each reservoir. (F) Apply the samples and cover the reservoirs using the tape.

Colorimetric Detection

Colorimetric detection method is known as an easy to use low cost qualitative method, which has been widely implemented in genomic detection systems. The color of hydroxynaphtol blue (HNB) has been changed from purple to blue during the amplification [8]. LAMP reaction makes significant amount of insoluble form of magnesium pyrophosphate. Consequently, concentration of the Mg²⁺ ions decreases during LAMP reaction progress. The color of HNB in the presence of the Mg²⁺ ions is purple and during progress of LAMP reaction the color has been changed into the blue due to reduction of the Mg²⁺ ions. This trend can be seen exactly during use of calcein as the colorimetric detection once the color of the dye changes from yellow to green.

Results and Discussion

In the development of the cassette, the roll was first installed. The samples were added to each reservoir and the whole roll was covered by a tape to protect the samples. We have used freshly prepared E. coli bacteria as a target for amplification. The E. coli was cultured overnight for 12 hours 5 μl of E. coli bacteria with different concentration was mixed with 20 μl of LAMP master mix and was loaded into different reservoirs. The flexible substrate was wrapped around the heater reel to start amplification. The temperature provides enough thermal shock to lyse the bacteria [10]. After 1 hour amplification time, the plastic roll is expanded for further analysis. The result can be checked by naked eye. We have tested various concentrations of E. coli bacteria from 3×10⁸−30 CFU/ml. The level of detection was 30 cfu/ml in 1 hour amplification. FIG. 3 shows the assay result.

The LAMP amplicons can be analyzed using light spectroscopy absorbance measurement. We have used absorbance spectra measurements of the LAMP reaction solution including different Mg²⁺ concentration when HNB was used as a colorimetric indicator. The absorbance spectra of the solution including Mg²⁺ and HNB were measured ranging from 600 nm to 700 nm wavelengths. The absorbance of solutions are related to the Mg²⁺ ions in the range of 620 nm to 670 nm and it represents that 650 nm is a suitable wavelength to measure LAMP solution absorbance spectra.

It is demonstrated that we can detect 30 cfu/ml of E. coil bacteria without any purification step in an 1-hour amplification. This device provides high throughput, easy to fabricate, low cost molecular diagnostic, which can detect any gram-negative bacteria or any other pathogens.

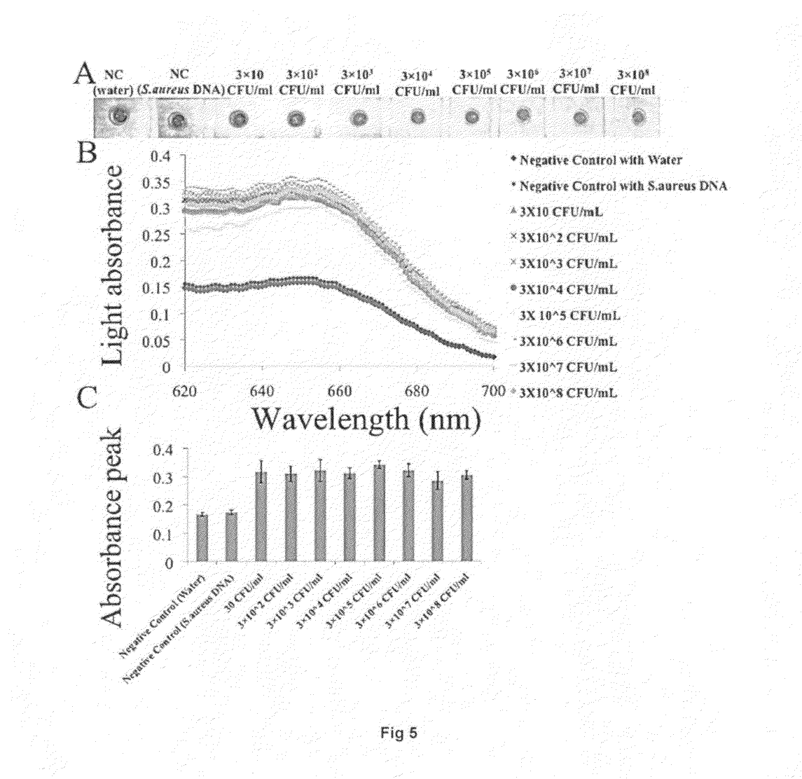

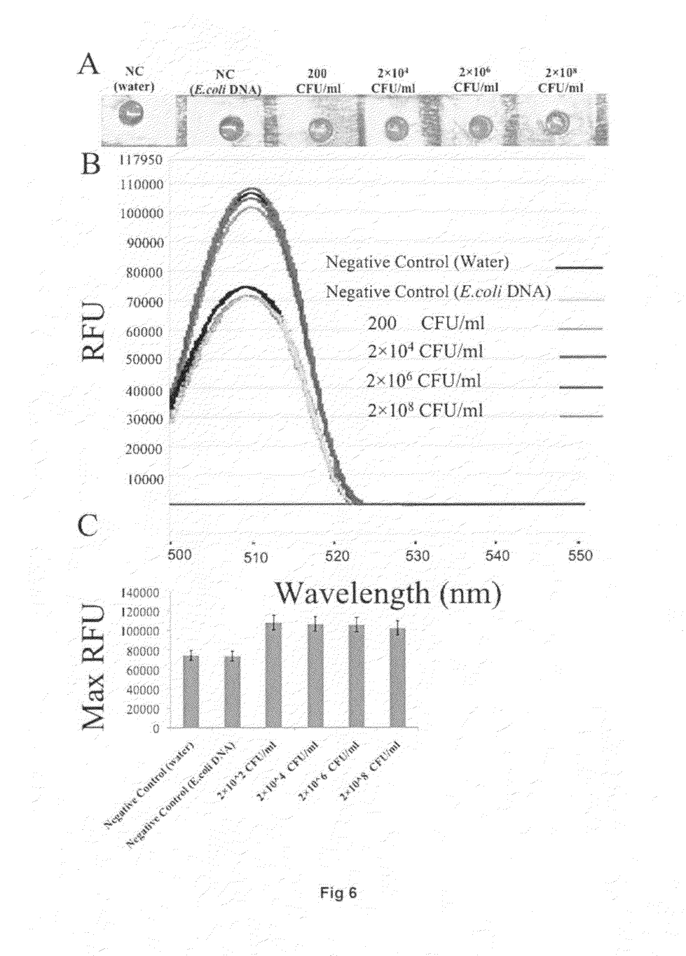

Referring to FIG. 5 results obtained from a colorimetric assay of E. coli bacteria on cassette are presented. (A) Color change of HNB has been observed due to existence of E. coli bacteria as a template and amplification of target gene in the samples. The color of negative control is purple. Other samples with different bacteria concentrations have been amplified. The colors of these samples are blue. (B) Light absorbency analysis of all samples. Negative control contained water. Samples were amplified for 1 hour. (C) Light absorbance of the samples at 650 nm wavelength. There is a significant gap in the light absorbance between the negative control and other samples, which reflects the fact that the purple color of the negative control absorbs less in comparison to the amplified samples with blue color. The level of detection is 30 cfu/ml.

Referring to FIG. 6, results obtained from colorimetric assay of S. aureus bacteria for different concentration are outlined. (A) Color of Calcein has been changed from yellow to the green. The color of negative control is yellow. (B) Florescence emission has been measured based on Relative Fluorescence Unit (RFU). The negative control sample is E. coli Bacteria. (C) Fluorescence intensity has been measured for various samples at 515 nm wavelength due to emitting of fluorescence in the amplified samples. The limit of detection is 200 CFU/ml.

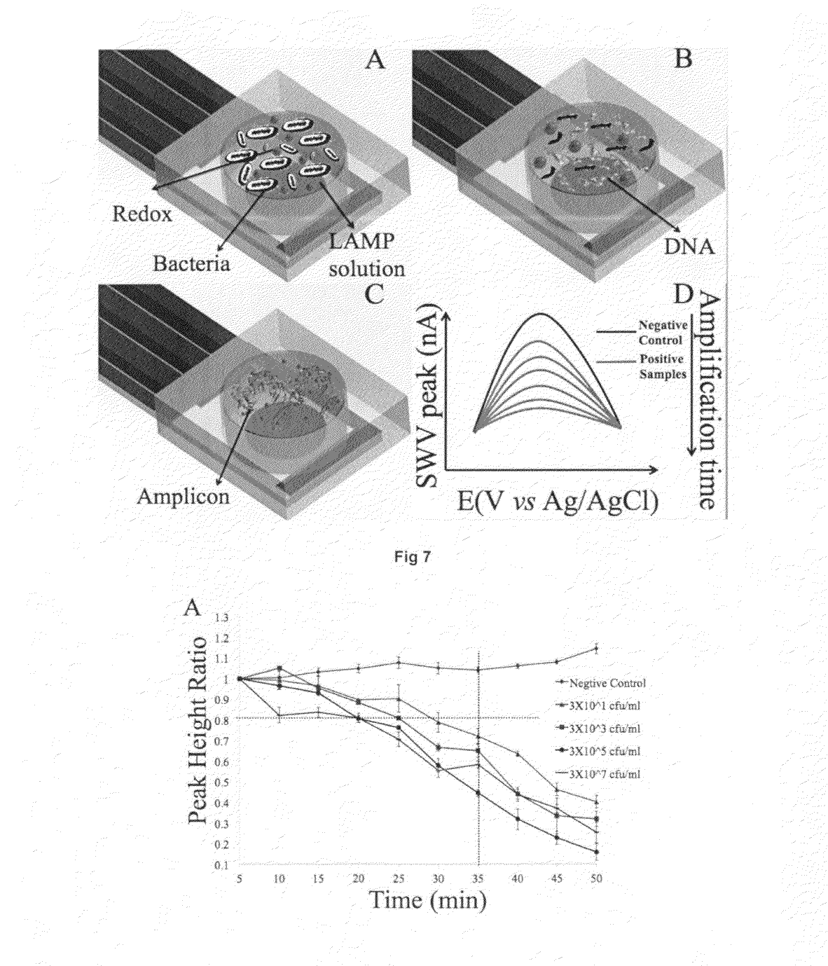

Referring to FIG. 7, schematic electrochemical real time monitoring of target amplicon. (A) Bacteria, redox molecules and LAMP solution before amplification. (B) Thermal shock lyses the bacteria and the DNA released in the solution (C) LAMP amplification process initiates and the redox molecules intercalates with the amplicons (D) Redox interaction with amplicon is shown in the electrochemical measurements using SWV. The oxidation peak is reduced due to the fact that more redox molecules binds with the amplicons and less redox molecules are oxidized during amplification process.

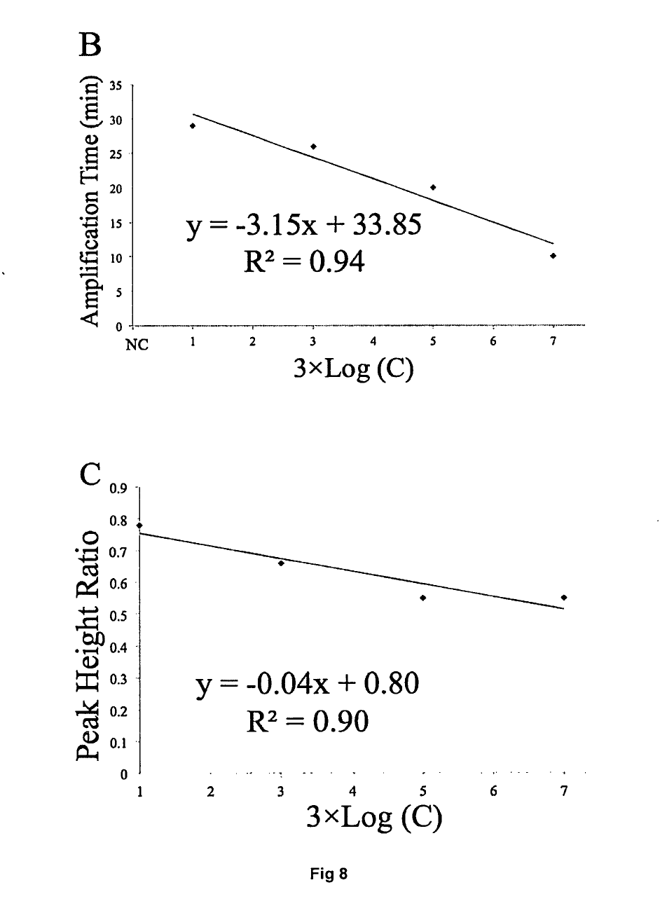

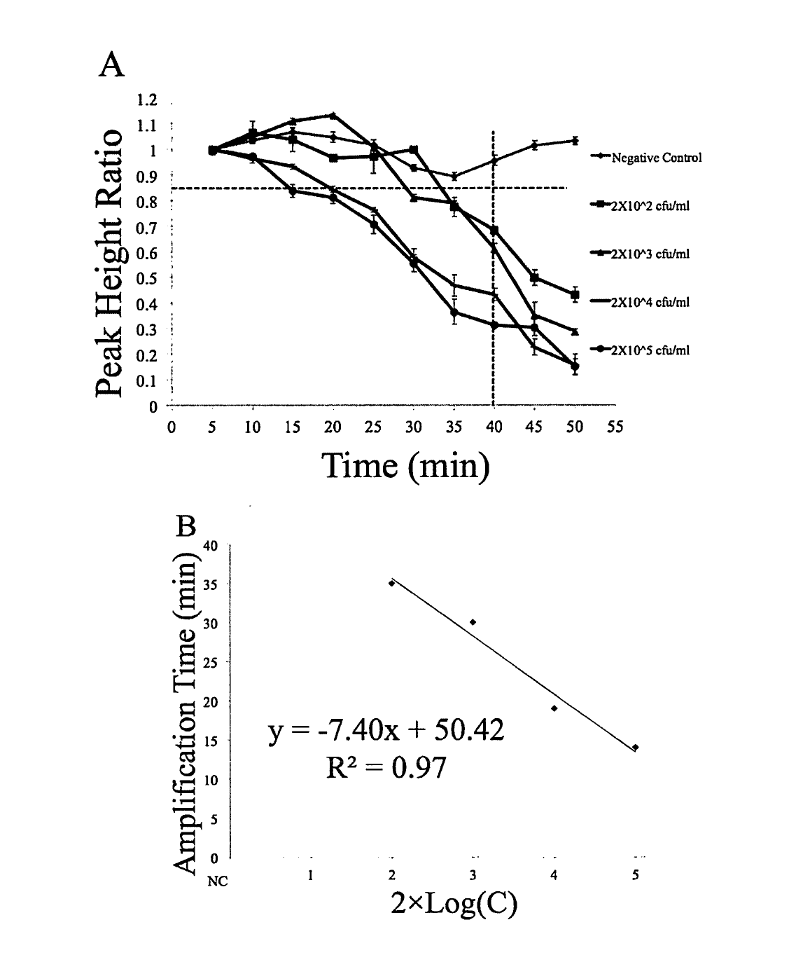

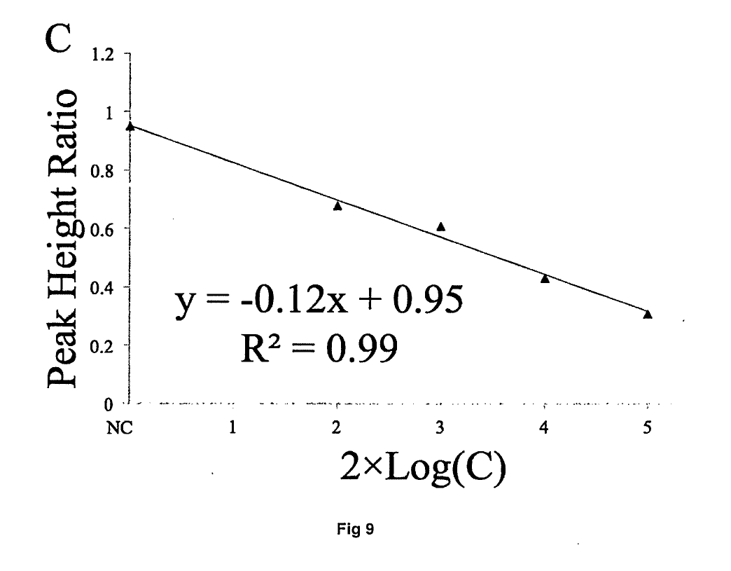

Referring to FIG. 8, results obtained from real time electrochemical assays in a flexible substrate are presented. Real time measurement of E.coli bacteria detection using 0.5 μM Os redox. (A) Peak height ratio of SWV vs amplification time. Different bacteria concentration has been used as a template and each 5 min electrochemical measurement has been implemented. Threshold value was set to 0.8. (B) Amplification time vs logarithmic concentration of E.coli bacteria. 10¹ cfu/ml of bacteria has been detected in 29 min. The regression fitted line and R̂2 is 0.94. (C) Quantification of E. coli bacteria in 35 min amplification based on peak height ratio. The fitted line R̂2 is 0.88.

Referring to FIG. 9, Real time measurement of S. aureus bacteria detection. (A) Real time monitoring of LAMP amplicon using Osmium redox detection. Various S. aureus bacteria concentration has been detected in a real time manner and each 5 min electrochemical measurement has been scanned till 50 min. The threshold value was set at 0.85. (B) Amplification time vs concentration of S. aureus bacteria. 35 min amplification time is required for detection of 200 cfu/ml of S. aureus. R² for the fitted line is 0.97. (C) Quantification of the S. aureus bacteria based on peak height ratio. R² for the fitted line is 0.99.

Conclusion

The inventors have designed and manufactured a cassette as a novel POC diagnostic device. The device's structure and operation have been demonstrated. Fabrication process of the flexible substrate has been shown. We could detect 30 cfu/ml of E. coli bacteria as well as 200 CFU/ml of Staphylococcus aureus DNA in one hour using colorimetric assay. This device can analyze many samples at the same time and is high throughput and can be used as POC in high demanded sample processing centers such as hospitals, doctors' offices or any other environment where it is desired to monitor the presence of pathogens.

The present invention can detect pathogens in a electrochemical real-time manner.

Although the present invention has been described hereinabove by way of specific embodiments thereof, a skill person will understand that it can be modified without departing from the spirit and nature of the invention as defined in the appended claims.

The description refers to a number of documents, the content of which is herein incorporated by reference in their entirety.

### REFERENCES

[1] A. V. Govindarajan, S. Ramachandran, G. D. Vigil, P. Yager and K. F. Bohringer “A low cost point-of-care viscous sample preparation device for molecular diagnosis in the developing world; an example of microfluidic origami” Lab on a Chip, vol. 12, pp. 174-181, 2012.

[2] M. Zourob , S. Elwary , A. Turner, “Principles of bacterial detection: biosensors, recognition receptors, and microsystems”. New York, N.Y.,USA: Springer; 2008.

[3] C. D. Chin , V. Linder and S. K. Sia “Lab-on-a-chip devices for global health: Past studies and future opportunities,” Lab on a Chip, vol. 7, pp. 41-57, 2007.

[4] J. B. Kaper , J. P. Nataro and H. L. T. Mobley , “Pathogenic Escherichia Coli,” Nature Reviews Microbiology, vol. 2, pp. 123-140, 2004.

[5] S. C. Clarke, “Diarrhoeagenic Escherichia coli—an emerging problem?,” Diagnostic Microbiology and Infectious Disease, vol. 41, pp. 93-98, 2001.

[6] C. F. Marrs , L. Zhang , B. Foxman, “Escherichia coli mediated urinary tract infections: Are there distinct uropathogenic E. coli (UPEC) pathotypes?,” FEMS Microbiology Letters, vol. 252, pp. 183-190 2005.

[7] P. J. Asiello, A. J. Baeumner “Miniaturized isothermal nucleic acid amplification, a review,” Lab on a Chip, vol. 11, pp. 1420-1430, 2011.

[8] M. Goto , E. Honda, A. Ogura , A. Nomoto and K. Hanaki, “Colorimetric detection of loopmediated isothermal amplification reaction by using hydroxy naphthol blue,” BioTechniques, vol. 46, pp. 167-172, 2009.

[9] M. U. Ahmed, S. Nahar, M. Safavieh, M. Zourob, “ Real-time electrochemical detection of pathogen DNA using electrostatic interaction of a redox probe” Analyst, vol 138, pp. 907-915, 2013.

[10] M. Safavieh, M. U. Ahmed, M. Tolba, and M. Zourob, “Microfluidic Electrochemical Assay for Rapid Detection and Quantification of Escherichia Coli,” Biosensors and Bioelectronics, vol. 31, pp. 523-528, 2012.

1. A portable device for detecting pathogens in a sample, wherein the detection is based on an assay involving one or more reagents and heat.

2. A portable device for detecting pathogens in a sample, comprising a flexible substrate having a plurality of spaced apart reservoirs along its length, each reservoir adapted for receiving reagents and the sample, the substrate being secured to a pair of spaced apart reels and movable there between by rolling, the device further comprising heating means, preferably secured to one of the two reels, for transmitting heat to the reservoirs, wherein the detection is based on an assay involving the reagents and heat.

3. A device for detecting pathogens in a sample, comprising a flexible substrate having a plurality of spaced apart reservoirs along its length, each reservoir having one or more reagents therein and adapted for receiving the sample, the substrate being secured to a pair of spaced apart reels and movable there between by rolling, the device further comprising heating means, preferably secured to one of the two reels, for transmitting heat to the reservoirs, wherein the detection is based on an assay involving the reagents and heat.

4. A device according to any one of claims 1 to 3, wherein the assay is selected from loop-mediated isothermal amplification (LAMP) assay, colorimetric assay, electrochemical assay, fluorescence-based assay and assay involving viscosity measurement.

5. A device according to any one of claims 1 to 4, wherein the assay allows for detection of amplification in real time.

6. A device according to any one of claims 1 to 5, wherein the pathogen is gram-negative or gram-positive bacteria, a virus, fungi or any other genomic DNA, RNA or ssDNA.

7. A device according to any one of claims 1 to 6, wherein the pathogen is Escherichia coli or Staphylococcus aureus.

8. A device according to any one of claims 1 to 7, wherein the reagent comprises LAMP reagent, hydroxynaphtol blue (HNB), calcein, a redox molecule such as osmium or ruthenium complexes.

9. A device according to any one of claims 1 to 8, which allows for high throughput detection of pathogens.

10. A device according to any one of claims 1 to 9, wherein the sample is from a human, an animal, food, a food environment, an office environment or any other environment.

11. A device according to any one of claims 1 to 10, further comprising heat control means for controlling heat transmitted to the reservoirs.

12. A device according to any one of claims 1 to 11, wherein heat transmitted to the reservoir is collected from other sources such as sun, solar-cells or any other heat-generating materials or systems.

13. A device according to any one of claims 2 to 12, wherein the reservoirs are closed reservoirs.

14. A device according to any one of claims 2 to 13, wherein the flexible substrate including the closed reservoirs constitutes a unitary body, or the flexible substrate further comprises a separate piece for covering the reservoirs.

15. A device according to claim 14, further comprising means for providing the separate piece for covering the reservoirs.

16. A device according to claim 15, wherein the means for providing the separate piece for covering the reservoirs comprises a reel system which has one or more reels.

17. A device according to any one of claims 2 to 16, wherein the flexible substrate is made of a material which is a plastic material made of polyethylene or any other plastic materials.

18. A device according to any one of claims 14 to 17, wherein the separate piece for covering the reservoirs is made of a plastic material made of polyethylene, poly acetate, polyvinylchloride (PVC) or polyethylene terephthalate (PET).

19. A device according to claim 15 or 16, wherein the separate piece is a cello-tape.

20. A device according to any one of claims 2 to 19, which is run using sun, solar cells or any other heat-generating materials or systems.

21. A method for diagnosing a disease in a human or an animal, comprising using the device according to any one of claims 1 to 20, wherein the sample is from the human or animal.

22. A method according to claim 21, wherein sample processing such as cell lysis or genetic materials fishing is performed prior to the use of the device.

23. A method according to claim 21 or 22, wherein the disease is a disease caused by the pathogens.

24. A method according to any one of claim 21 to 23, wherein the disease is meningitis, urinary tract infection (UTI), a medical condition caused by food poisoning, biological contamination or bioterrorism.
